(19) United States
(12) Patent Application Publication
(10) Pub. No.: US 2016/0376640 A1
(43) Pub. Date: Dec. 29, 2016

(54) DETERMINATION OF EXOSOME PURITY METHODS AND APPARATUS

(71) Applicant: Beckman Coulter, Inc., Brea, CA (US)

(72) Inventors: Carley Denise Ross, Fort Collins, CO (US); Thomas Hans Ramin, Fort Collins, CO (US)

(21) Appl. No.: 15/190,632

(22) Filed: Jun. 23, 2016

Related U.S. Application Data
(60) Provisional application No. 62/184,682, filed on Jun. 25, 2015.

(51) Int. Cl. C12Q 1/68; G01N 21/64; G01N 21/33

(52) U.S. Cl. CPC C12Q 1/6818; C12Q 1/6804; G01N 21/33; G01N 21/6428; G01N 2021/6439

(57) ABSTRACT

Methods and apparatuses for determining exosome purity are disclosed. In an example embodiment, a laboratory instrument apparatus includes an analytical centrifuge configured to rotate a solution causing a plurality of particles to separate. The analytical centrifuge is also configured to perform an interference measurement on the solution and/or three absorbance measurements on the solution. A computer processor communicatively coupled to the analytical centrifuge is configured to determine an exosome purity of the solution based on the three absorbance measurements and/or the interference measurement.

### PRIORITY CLAIM

The present application claims priority to and the benefit of U.S. Provisional Patent Application No. 62/184,682, filed on Jun. 25, 2015, the entirety of which is incorporated herein by reference.

### BACKGROUND

Presently, there is a growing interest in extracellular vesicles (“EVs”), which include microparticles, apoptotic bodies, and exosomes. EVs are cell-derived vesicles that are released from plasma membrane of cells. EVs have specialized functions to assist in, for example, coagulation, intercellular signaling, and waste management. Research within the last five years has shown that EVs can potentially be used to diagnose disease, determine disease prognosis, or determine health. EVs may also be used in therapy to treat some diseases.

Exosomes, a subset of EVs, have a relatively small size (between 30 and 100 nanometers (“nm”)) that makes them especially difficult to measure in a laboratory environment. By comparison, microparticles of a cell's plasma membrane have a size between 100 nm to 1000 nm, thereby making them much easier to identify and analyze. Historically, differential centrifugation has been the primary method for isolating exosomes. More recently, additional technologies such as filtration and polymer precipitation have also been used to isolate exosomes. Additionally, recent advances in flow-cytometric detection now enable nanoparticles to be detected at the higher end of the exosome size range. For instance, the Malvern® NanoSight instrument uses a scatter profile for exosome detection. Other known flow-cytometry instruments use dynamic light scattering (“DLS”) for exosome detection. The scatter profile produced by these known instruments provides an indistinct ‘smear’ to determine a size distribution of exosome within a solution. While providing a relatively fast analysis, known scatter and smear-based analysis have a low resolution (e.g., 3:1 resolution) and cannot differentiate between proteins, lipids, and nucleic acids.

### SUMMARY

The example methods and apparatuses disclosed herein are configured to determine an exosome purity using an interference measurement and/or one or more absorbance measurements on particles separated within a solution using an analytical centrifuge. The interference measurement and/or the one or more absorbance measurements enable the detection of lipid content, nucleic acid content, and/or protein content within a sample solution. The presence of lipid content in addition to protein or nucleic content indicates a presence of exosomes. A purity of the exosomes may be determined from a ratio of the lipid, protein, and nucleic acid content. As disclosed herein, the use of an interference measurement and/or an absorbance measurement in an analytical centrifuge enables the detection and characterization of exosomes that are as small as 20 nm in diameter.

In an example embodiment, a laboratory instrument includes an analytical centrifuge configured to rotate a solution causing a plurality of particles to separate. The analytical centrifuge also performs at least one of i) an interference measurement on the solution, and ii) three absorbance measurements on the solution. The laboratory instrument also includes a computer processor communicatively coupled to the analytical centrifuge configured to determine an exosome purity of the solution based on the at least one of the three absorbance measurements and the interference measurement.

In another example embodiment, a method of characterizing extracellular vesicles in a sample solution includes rotating the sample solution using an analytical centrifuge to separate the sample solution into a plurality of fractions. The method also includes measuring with the analytical centrifuge a first interaction of the plurality of fractions with a first energy beam and a second interaction of the plurality of fractions with a second energy beam. The method further includes characterizing, via a computer processor, the extracellular vesicles along the plurality of fractions based on the first interaction and the second interaction.

Additional features and advantages of the disclosed system, method, and apparatus are described in, and will be apparent from, the following Detailed Description and the Figures.

### BRIEF DESCRIPTION OF THE FIGURES

FIG. 1 shows a diagram of an example laboratory analysis system, according to an example embodiment of the present disclosure.

FIG. 2 shows a diagram of forces on particles or fractions when a solution within an analytical cell is being spun by a rotor of an analytical centrifuge, according to an example embodiment of the present disclosure.

FIG. 3 shows a diagram of absorbance measurements on an analytical cell over a period of time to determine sedimentation velocity, according to an example embodiment of the present disclosure.

FIG. 4 shows a diagram of a laboratory instrument and a computer processor of the example laboratory analysis system of FIG. 1, according to an example embodiment of the present disclosure.

FIG. 5 shows a diagram of the laboratory instrument of FIGS. 1 and 4 configured to perform an absorbance measurement, according to an example embodiment of the present disclosure.

FIGS. 6 to 12 show diagrams of example absorbance measurements performed by the laboratory instrument of FIGS. 1, 4, and 5 and analyses of the absorbance measurements performed by the computer processor of FIGS. 1 and 4, according to example embodiments of the present disclosure.

FIG. 13 shows a diagram of the laboratory instrument of FIGS. 1 and 4 configured to perform an interference measurement, according to an example embodiment of the present disclosure.

FIGS. 14 to 16 show diagrams of example interference measurements performed by the laboratory instrument of FIGS. 1, 4, and 13 and analyses of the interference measurements performed by the computer processor of FIGS. 1 and 4, according to example embodiments of the present disclosure.

FIG. 17 illustrates a flow diagram showing example procedures to determine exosome purity, according to an example embodiment of the present disclosure.

FIGS. 18 and 19 show diagrams of a characterization of HeLa exosomes isolated in a solution using flow-cytometry sorting, according to an example embodiment of the present disclosure

### DETAILED DESCRIPTION

The present disclosure relates in general to characterizing exosomes, and in particular, to using more than one analytical centrifuge measurement to determine a purity of EVs or exosomes in a sample solution. The example method and apparatus disclosed herein uses an analytical centrifuge (or analytical ultracentrifuge (“AUC”)) to perform multiple measurements on a sample solution to determine EV or exosome purity at a relatively high resolution. Experiments have shown that the example methods and apparatus disclosed herein detect exosomes ranging in diameter between 20 nm and 130 nm. In contrast known methods and instruments are only able to detect exosomes having diameters greater than 50 nm. Further, the example method and apparatus disclosed herein provide an exosome resolution of 10 nm for 100 nm exosomes.

As disclosed in more detail below, the example methods and apparatus perform at least two measurements on a sample solution that is being rotated within an analytical centrifuge. The measurements may include one or more interference measurements using a laser, one or more absorption measurements using light with a wavelength between 190 nm and 800 nm, preferably between 190 nm and 300 nm, and/or one or more absorbance measurements using fluorescence markers and light with a wavelength between 400 nm and 700 nm, preferably between 450 nm and 600 nm. In an embodiment, one interference measurement and up to three absorbance measurements from 190 nm to 800 nm are performed on each sample with a total of seven samples being measured and analyzed simultaneously within an analytical centrifuge. The seven samples may have the same or different compositions. The multiple measurements enable a sedimentation velocity, concentration, mass, mass distribution, and radius (e.g., Stokes Radius) to be determined for a plurality of particles or fractions within the solution. Additionally, the multiple measurements enable a species specific composition to be determined based on the detection of lipid content, protein content, and nucleic acid content. As discussed above, known instruments are unable to determine specific species compositions.

The example systems and methods are disclosed in conjunction with the characterization of EVs or exosomes. It should be appreciated that the example system and method may be used to characterize larger biological particles including microvesicles, endosomes, vesicles, ribosomes, vacuoles, cytosols, lysosomes, centrosomes, protein, lipids, nucleic acid, oligomers, aggregates, colloids, small structures, etc. It should also be appreciated that the example method and apparatus disclosed herein may be used to characterize chemical particles including quantum dots, polymer nanoparticles, carbon nanotubes, pharmaceuticals, industrial fluids, environmental fluids, etc.

Reference is made herein to analytical centrifuges. As described in more detail below, an analytical centrifuge includes any centrifuge or AUC that is configured to analyze a solution located within one or more analytical cells that are spun by a rotor. The analytical centrifuge is configured to rotate the one or more analytical cells at a rate between 1,000 revolutions per minute (“RPM”) and 60,000 RPM to cause sedimentation of particles within the solution. The analytical centrifuge is configured to perform one more measurements on the sedimentation using one or more light beams over the course of one or more rotational speeds. Data from the measurements is used to determine characteristics of the particles, thereby enabling the determination of species type, purity, mass, and mass distribution.

### Example Analysis System

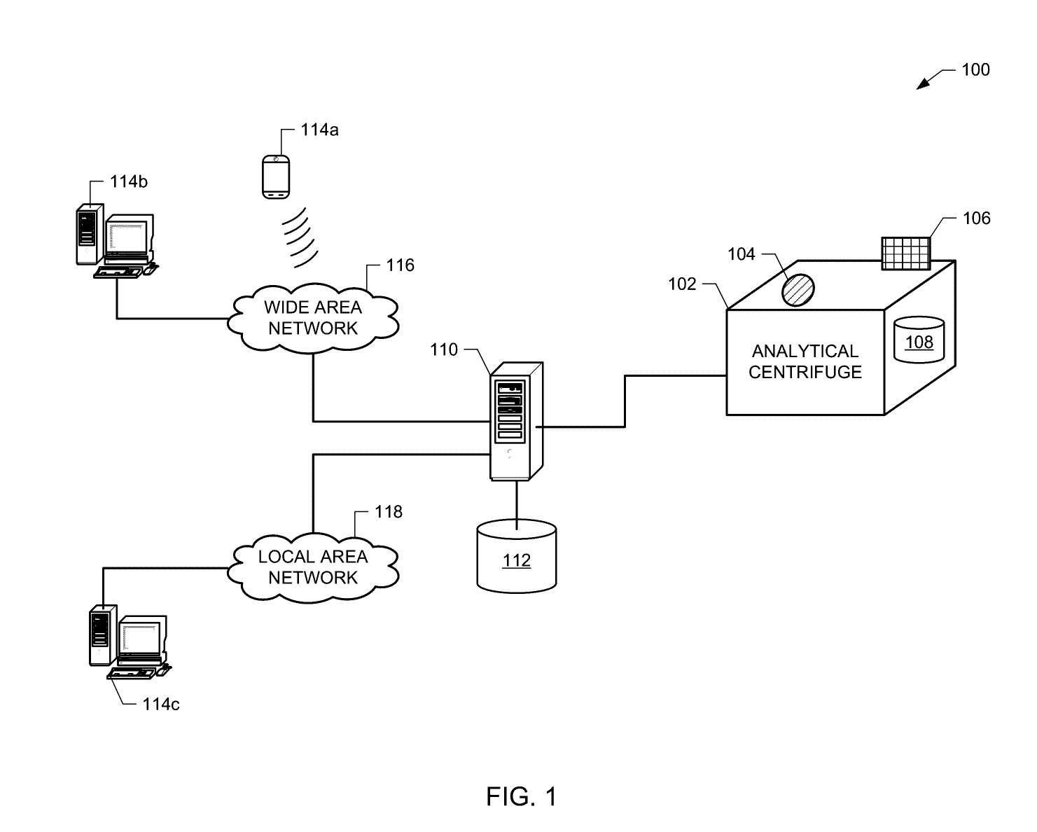

FIG. 1 shows a diagram of an example laboratory analysis system 100, according to an example embodiment of the present disclosure. The example analysis system 100 includes at least one laboratory instrument 102 configured to perform a measurement on a solution that includes a plurality of particles or fractions (e.g., exosomes). The laboratory instrument 102 may be an analytical centrifuge or AUC such as the Beckman Coulter® AUC ProteomeLab™ XL-A/I. The example laboratory instrument 102 includes a chamber 104 having a rotor configured to rotate one or more analytical cells that contain a solution to be measured or analyzed.

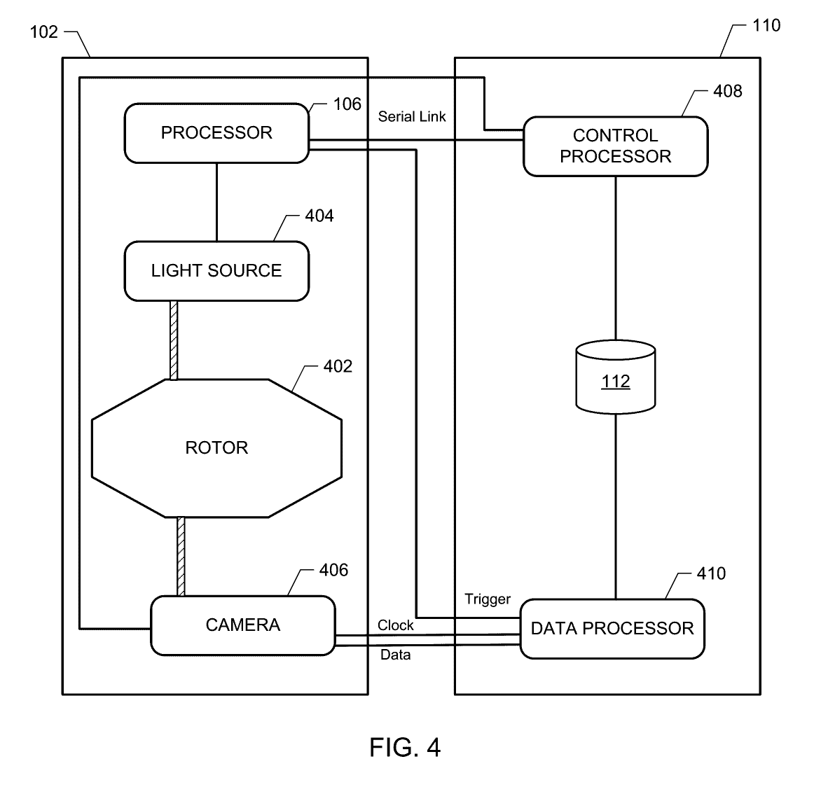

FIG. 4 shows a diagram of a rotor 402 within the laboratory instrument 102. The rotor 402 may be configured to rotate the analytical cells between 1,000 RPM and 60,000 RPM for a specified time. Typical rotation speeds for absorbance and interference measurements are between 20,000 RPM and 30,000 RPM. The relative centrifugal force exerted on the solution within the cells by the rotor 402 may range from 100 g-force (“g's”) to 300 g's. The rotor 402 may accommodate four, six, eight, or more analytical cells and/or one or more counterbalances. Each analytical cell may contain between 100 microliters (“μl”) and 500 μl, preferably about 400 μl of solution.

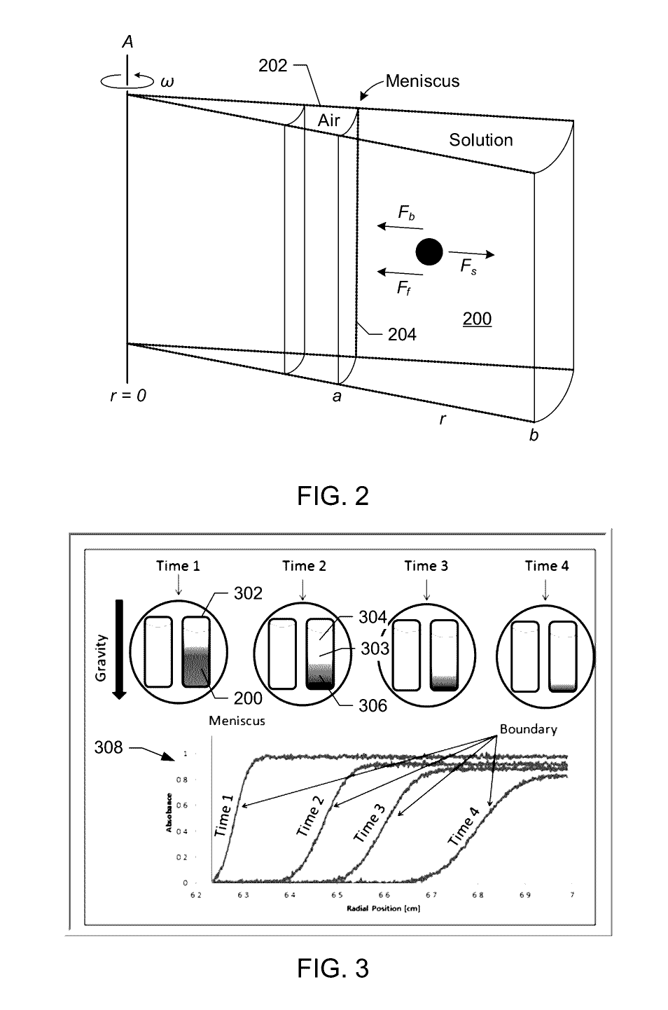

FIG. 2 shows a diagram of forces on particles or fractions when a solution 200 within an analytical cell is being spun by the rotor 402. As illustrated, for any given analytical cell, a sedimenting, centrifugal, or gravitational force (Fₛ) causes the solution 200 to accumulate away from an axis of rotation (A). An air gap 202 may form in the space vacated by the solution 200, with a meniscus 204 forming in the solution 200 at a boundary with the air gap 202. The gravitational force (Fₛ) on a particle is determined by the distance (or radius) of the particle from the axis of rotation, r, and the square of the angular velocity, co (in radians per second), and may be determined from equation (1) below.

$${F}_{s} = m {ω}^{2} r = \frac{M}{N} {ω}^{2} r \qquad ( 1 )$$

In equation (1), m is the mass in grams of a single particle, M is the molar weight of the solute in g/mol, and N is Avogadro's number (approximately 6.02214179×10²³ mol⁻¹). Note that the molecular weight is numerically equal to the molar weight, but is dimensionless.

In addition to the gravitational force, particles within the solution 200 are subject to a buoyant force (F_{b}) and a frictional force (F_{ƒ}). The buoyant force is equal to the weight of the solution displaced while the frictional force is equal to a frictional drag on a particle, which is proportional to the particle's velocity. The buoyant force may be determined using equation (2) below, where m₀ is the mass of fluid displaced by the particle. m₀ may be determined using equation (3) below, where ν̅ is the volume in mL that each gram of the solute occupies in the solution 200 (the partial specific volume, which is the inverse of its effective density) and ρ is the density of the solvent (g/mL).

$$\begin{matrix}{F}_{b} = - {m}_{0} {ω}^{2} r & ( 2 ) \\ {m}_{0} = m \overline{v} ρ = \frac{M}{N} \overline{v} ρ & ( 3 )\end{matrix}$$

When a density of a particle is greater than that of the solvent within the solution 200, the particle will begin to sediment. As the particle begins to move along a radial path towards the “bottom” (e.g., edge or side) of an analytical cell, its velocity increases as its radial distance r increases. Particles moving through a viscous fluid experience a frictional force F_{ƒ} that is proportional to the velocity, which may be expressed as equation (4) below. ƒ is the frictional coefficient, which depends on the size and shape of the particle, and ν is the velocity of the particle.

F_{ƒ}=−ƒν (4)

A sedimentation coefficient of the particle (or a group of particles of the same species) may be determined using gravitational force Fₛ, the buoyant force F_{b}, and the frictional force F_{ƒ}. During rotation, a particle will achieve a velocity such that the total force (i.e., gravitational force Fₛ, the buoyant force F_{b}, and the frictional force F_{ƒ}) is equal to zero. Equations (2) to (4) may be summed to equal zero and rearranged to place molecular parameters on one side of the equation and experimentally measured parameters on the other side to achieve equation (5), which is shown below.

$$\frac{M ( 1 - \overline{v} ρ )}{Nf} = \frac{v}{{ω}^{2} r} ≡ s \qquad ( 5 )$$

In equation (5), s is the sedimentation coefficient, which is equal to the experimentally measured side (right side) and the molecular parameters on the left side. The sedimentation coefficient may be expressed as seconds, with typical values having a magnitude of 1×10⁻¹³ seconds (i.e., 100 femtoseconds), which is commonly called 1 Svedberg (S). This relation enables the sedimentation coefficient to be determined from particle velocity (ν), angular velocity (ω), and the radius from the axis of rotation (r), which are known (or can be measured using absorbance or interference). For instance, the angular velocity (ω) is directly related to a rotational speed of the rotor 402. The sedimentation coefficient is proportional to the molecular weight (M) multiplied by a buoyancy factor (1−ν̅ρ) and inversely proportional to the frictional coefficient. Large values of S (e.g., a faster sedimentation rate) correspond to larger molecular weight. Accordingly, denser particles tend to sediment more rapidly while elongated proteins, for example, have larger frictional coefficients and sediment more slowly.

As mentioned above, the particle velocity (ν) may be measured using absorbance or interference. The particle velocity (ν) for an individual particle is very difficult to determine. Therefore, the particle velocity (ν) is determined by measuring how a sedimentation boundary of the solution moves over time, which indicates how quickly a group of the same particles is moving through the solution 200. The speed of the sedimentation boundary is referred to as sedimentation velocity. FIG. 3 shows a diagram of absorbance measurements on an analytical cell over a period of time to determine sedimentation velocity. An analytical cell 302 includes the solution 200, which over time separates into particles or fractions that accumulate at a bottom or edge. A sedimentation boundary 303 forms between a depletion region 304 and a concentration region 306 of the solution 200. A graph 308 shows how a position of the sedimentation boundary 303 changes over time, which is measured through an absorbance measurement. The sedimentation velocity can accordingly be determined based on how the sedimentation boundary 303 changes over time, which is equal to how a concentration of particles within the solution move through a radius of the test cell

The particle velocity (ν) is accordingly equal to dr_{b}/dt, where r_{b} is the radius of the sedimentation boundary. This relation may be substituted into equation (5) and integrated over time to produce equation (6), where r_{b}(t) is the position of the boundary at time t. Graph 308 shows ln[r_{b}(t)/r_{b}(t₀)] versus (t−t₀) (for the four times) provides ω²s, which enables s to be determined.

$$ln \frac{{r}_{b} ( t )}{{r}_{b} ( {t}_{0} )} = {ω}^{2} s ( t - {t}_{0} ) \qquad ( 6 )$$

The sedimentation velocity enables other characteristics of particles or fractions to be determined. These characteristics include mass, mass distribution, and Stokes Radius. Additionally, measurements of sedimentation velocity over different light wavelengths enables species specific compositions to be determined by identifying, for example, lipid content, protein content, and nucleic acid content.

As discussed in more detail below, the example laboratory instrument 102 of FIGS. 1 and 4 may also include one or more light sources 404. The type of light source is based on the measurement being performed. For instance, the light source 402 may be configured to transmit light of a particular wavelength for absorbance measurements. In these instances, the light source 402 may include a Xenon flash tube configured to produce intense, incoherent, full-spectrum white light for short durations of time to generate light wavelengths from 190 nm to 800 nm. Alternatively, for interference measurements, the light source 402 may include a diode laser.

As illustrated in FIGS. 1 and 4, the example laboratory instrument 102 includes a processor 106 configured to control operation of the rotor 402, the light source 404, and a camera 406 to perform absorbance measurements and/or interference measurements. The processor 106 is in communication with a memory 108 that stores computer-readable instructions that specify how measurements are to be performed according to one or more specified routines or algorithms. For example, the processor 106 may instruct the rotor 402 to operate at a specified speed and instruct the light source 404 to transmit light at a specified time based on the rotational speed of the rotor 402, thereby exposing a solution within an analytical cell to light as the analytical cell passes in front of the light source. The processor 106 may also coordinate the timing of the light source 404 with the data acquisition timing of the camera 406 such that the camera is activated during the time in which the light source 404 is activated. An algorithm or routine may specify a time duration for rotation and any changes to the rotor speed. In some instances, the processor 106 causes the rotor 402 to rotate according to a profile with different rotation speeds. The processor 106 may also instruct one or more light sensors or cameras (e.g., the camera 406) to record the light received through the cell, which amounts to performing a measurement on the sample solution. The example processor 106 may store the measurement data until a measurement test is completed. Alternatively, the processor 106 may transmit the data as the data is received.

The example camera 406 may include any photosensor capable of detecting light. For instance, the camera 406 may include a charged-coupled device (“CCD”), a high-definition camera, a three-dimensional camera, etc. The camera 406 is configured to record one or more images with a resolution, for example, of 96 pixels by 2048 pixels. In some instances, the camera 406 may record a series of images to form a video recording.

As illustrated in FIGS. 1 and 4, the example laboratory instrument 102 is communicatively coupled to a computer processor 110, which may include a control processor 408 and a data processor 410. The computer processor 110 may include any server, desktop computer, workstation, laptop, tablet computer, smartphone, etc. capable of processing and/or storing measurement data. The example control processor is configured to transmit one or more messages to the processor 106 of the laboratory instrument 102 regarding one or more measurements to be performed. For instance, a user may create or specify parameters (e.g., measurement type, number of analytical cells to be measured, light wavelength, test duration, measurement intervals, etc.) of a measurement to be performed using the control processor 408. After receiving the parameters, the control processor 408 transmits the parameters within one or more messages to the processor 106, which then operates the light source 404, the rotor 402, and the camera 406 to perform the measurement. Alternatively, the specification of parameters may be performed using the processor 106 of the laboratory instrument 102 and a user interface or display at the laboratory instrument 102.

The example data processor 410 is configured to receive, process, and analyze images recorded by the camera 406. The data processor 410 may also be configured to instruct the camera 406 when to record images. The computer processor 110 includes and/or is communicatively coupled to a memory 112 that stores computer-readable instructions accessible by the data processor 410 for analyzing the measurement data from the laboratory instrument 102. For example, the memory 112 may store Sedfit software (e.g., Sedfit version 14.6e) by Peter Schuck to analyze and visualize the measurement data from the laboratory instrument 102. The analysis of the measurement data may include the data processor 410 performing a determination of particle or fraction mass, a determination of mass distribution, and/or a determination of Stokes Radius. The data processor 410 may also determine species specific compositions by identifying, for example, lipid content, protein content, and nucleic acid content. In some instances, the memory 112 may store additional instructions for determining particle purity based on the analyzed measurement data. For example, instructions may be configured to determine exosome purity based on a ratio of lipid content, protein content, and nucleic acid content within a sample solution.

While FIG. 1 shows the computer processor 110 connected to only the laboratory instrument 102, it should be appreciated that the computer processor 110 may be communicatively coupled to additional or other laboratory instruments. The other laboratory instruments may include analytical centrifuges, AUCs, solution sorters, and particle analyzers. For example, the computer processor 110 may be connected to a Beckman Coulter® MoFlo® Astrios™ EQ sorter, Beckman Coulter® Optima™ XPN AUC, and/or the Beckman Coulter® DelsaMax Pro™.

The example computer processor 110 may include one or more interfaces to enable external user devices 114 to access the raw and/or analyzed measurement data stored at, for example, the memory 112. The user devices 114 may connect to the computer processor 110 via any wide area network 116 (e.g., the Internet) and/or local area network (“LAN”) 118. In some instances, the user devices 114 may use a virtual LAN (“VLAN”) or other secure tunnel to directly connect to the computer processor 110. The user devices 114 may include any smartphone, tablet computer, laptop computer, desktop computer, server, processor, etc.

### Absorption Measurement Embodiments

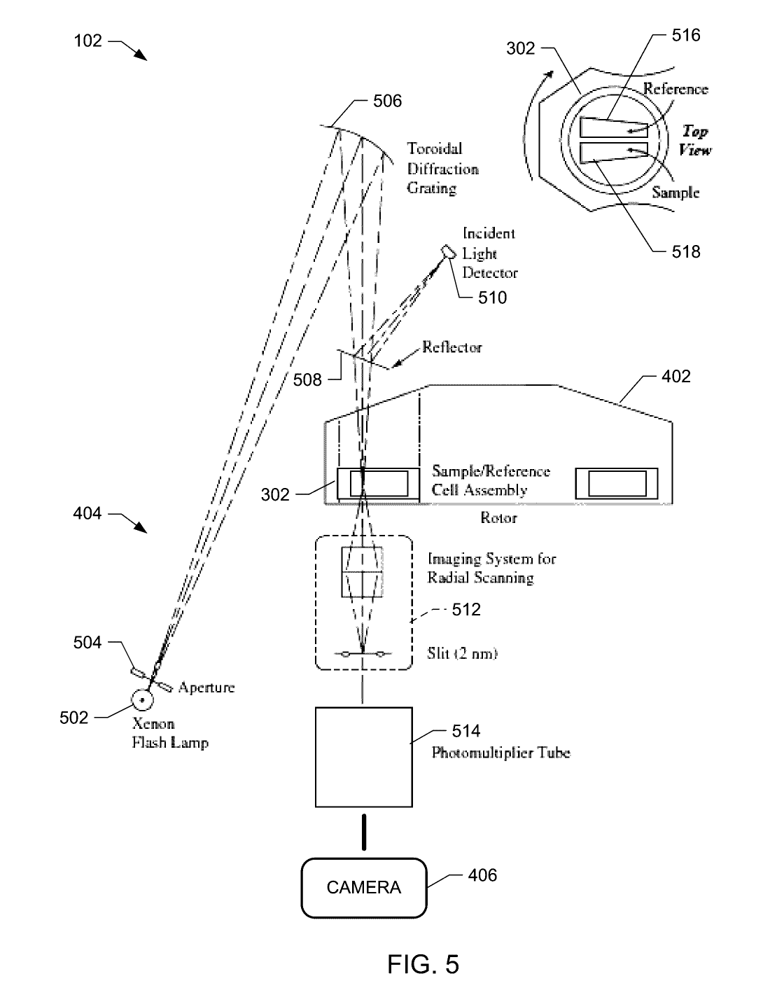

FIG. 5 shows a diagram of the laboratory instrument 102 of FIGS. 1 and 4 configured to perform an absorbance measurement, according to an example embodiment of the present disclosure. In this embodiment, an absorbance measurement is preformed by instructing the light source 404 to briefly produce a light with a wavelength that is absorbed or otherwise interacts with particles or fractions within a sample solution. The laboratory instrument 102 may perform a scan across a range of wavelengths to determine which wavelengths are absorbed, which indicates a species or components with a sample solution. Absorbance measurements may also be made over time to determine a sedimentation velocity.

As illustrated in FIG. 5, the light source 404 includes an Xenon flash lamp 502 configured to produce intense, incoherent, full-spectrum white light for short durations of time. The produced light may be transmitted through an aperture 504 to define a cone angle for the light incident on a toroidal diffraction grating 506. The example toroidal diffraction grating 506 includes a pattern of parallel lines to provide diffraction grating to split the light into different wavelengths. The toroidal diffraction grating 506 may be rotated by a motor to enable a selected wavelength to be transmitted to a sample solution.

The light source 404 also includes a reflector 508 configured to reflect a portion of the light to an incident light detector 510, which measures an amount of light being transmitted to the analytical cell 302. A moveable slit 512 is configured to select a portion of the light transmitted through the analytical cell 302 for recording by the camera 406. The slit 512 is moveable along a radius of the analytical cell 302 to determine, for instance, a location of the sedimentation boundary. A photomultiplier tube 514 is configured to multiple or amplify light received by the slit 512 to enable, for example, detection of single protons. The photomultiplier tube 514 may amplify light by as much as 10⁸. The camera 406 records the light amplified by the photomultiplier tube 514.

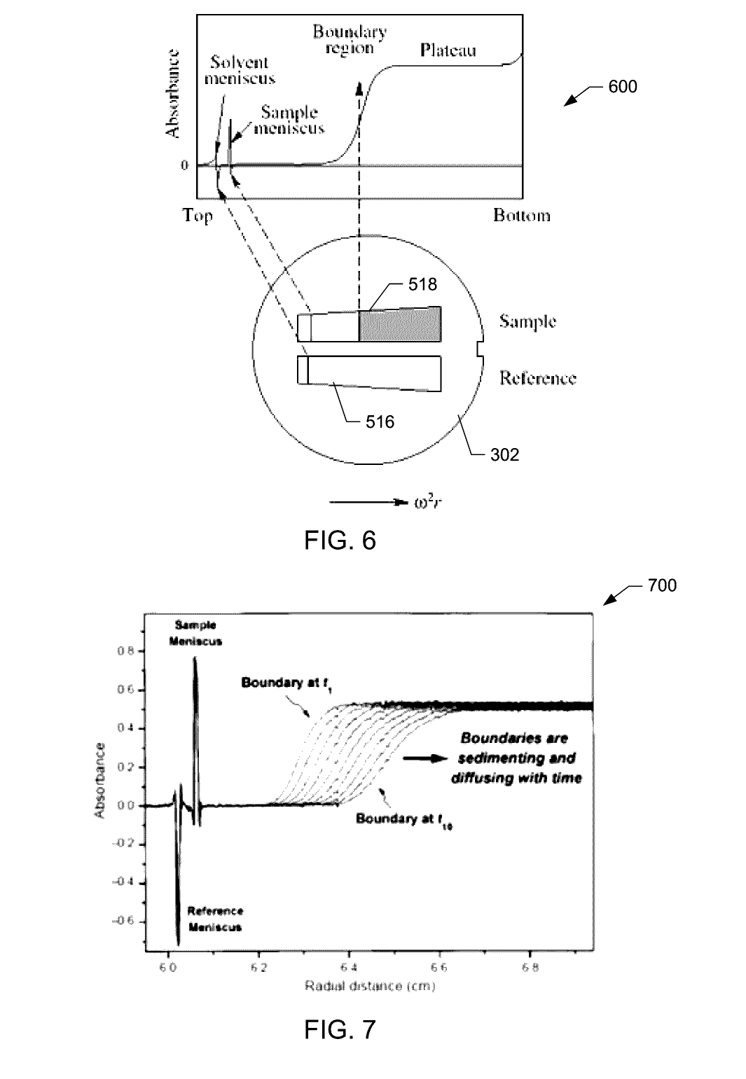

To perform an absorbance measurement, the example laboratory instrument 102 is configured to measure, for a particular wavelength, light incident through a reference side 516 and a sample side 518 of the analytical cell 302. The difference in light intensity between the reference side 516 and the sample side 518 is indicative of the absorbance at that wavelength. Separate absorbance measurements are performed across the length or radius of the analytical cell 302 (e.g., scanning from left-to-right of the analytical cell 302 shown in FIG. 5) to determine a profile of a concentration of the particles or fractions. FIG. 6 shows a diagram 600 of absorbance measurements performed across the length or radius of the analytical cell 302 at a first instance of time, according to an example embodiment of the present disclosure. As illustrated in FIG. 6, the absorbance increases at the boundary region, which is where the particles begin to concentrate or sediment. FIG. 7 shows a diagram 700 of absorbance measurements at different times (times 1 to times 10) for the analytical cell 302. As illustrated, the boundary region moves toward the outer edge of the analytical cell 302. The distance moved over time enables the sedimentation velocity to be determined. The data processor 410 differentiates the separate curves in FIG. 7 by time and fits the differentiation to a Gaussian curve. The data processor 410 determines a peak of the Gaussian curve, which is the sedimentation coefficient, and an area under the curve, which is the diffusion coefficient (or percent population (%).

As discussed above, the laboratory instrument 102 may be configured to perform one or more absorbance measurements at different light wavelengths to determine a species or composition of particles or fractions within a solution. For example, lipids have a typical absorbance around 210 nm, nucleic acids have a typical absorbance around 260 nm, and proteins have a typical absorbance around 280 nm. The content of a solution may be determined by performing absorbance measurements at 210 nm, 260 nm, and 280 nm. The data processor 410 may process the absorbance data from each measurement to determine if lipids, nucleic acids, or proteins are present. Conditioned on determining lipid content and at least one of nucleic acid content or protein content, the data processor 410 may determine the solution contains exosomes. The data processor 410 may also determine an amount of absorption at each wavelength to determine, for instance, a concentration or amount of each type of particle. For example, relatively high absorbance indicates a greater concentration of that type of particle. The data processor 410 may also (or alternatively) determine a sediment coefficient to determine a mass distribution for that type of particle. The data processor 410 may then determine ratios between lipids, nucleic acids, and proteins to determine, for example, an exosome purity and/or a presence of debris (such as cholesterol rafts).

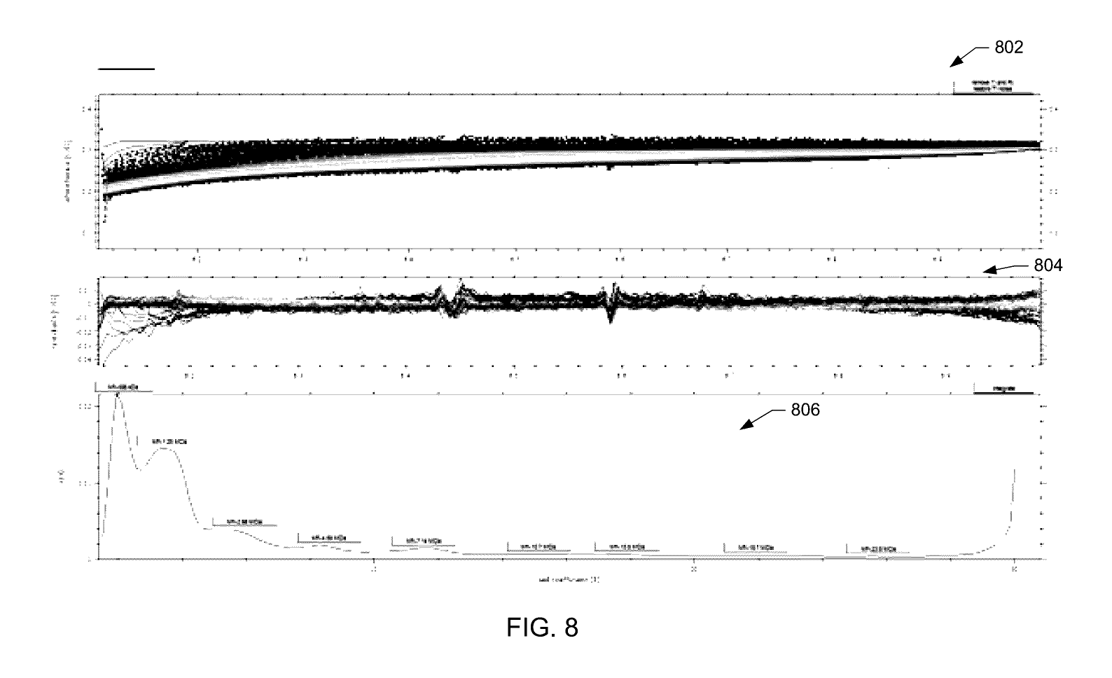

FIG. 8 shows a diagram of three graphs 802, 804, and 806 created by the data processor 410 based on absorbance measurements on a sample solution that includes MCF-7 exosomes, according to an example embodiment of the present disclosure. In this example, the processor 106 instructs the rotor 402 to rotate or spin at a rate of 25,000 RPM for 4 hours. The graphs 802, 804, and 806 are for absorbance measurements at 260 nm. Graph 802 shows absorbance of a sample solution with the MCF-7 exosomes at different time periods. The absorbance is displayed in relation to a radial distance within the analytical cell 302, which shows boundary migration over time, thereby enabling sedimentation velocity to be determined. Graph 804 shows residuals over the same radial distance. Graph 806 shows a Gaussian curve for the time differences of the lines in graph 802 compensating for the residuals shown in graph 804. The peaks of the curve in graph 806 correspond to the sedimentation coefficients while the area under the curve represents the mass distribution of the particles that absorbed light at 260 nm.

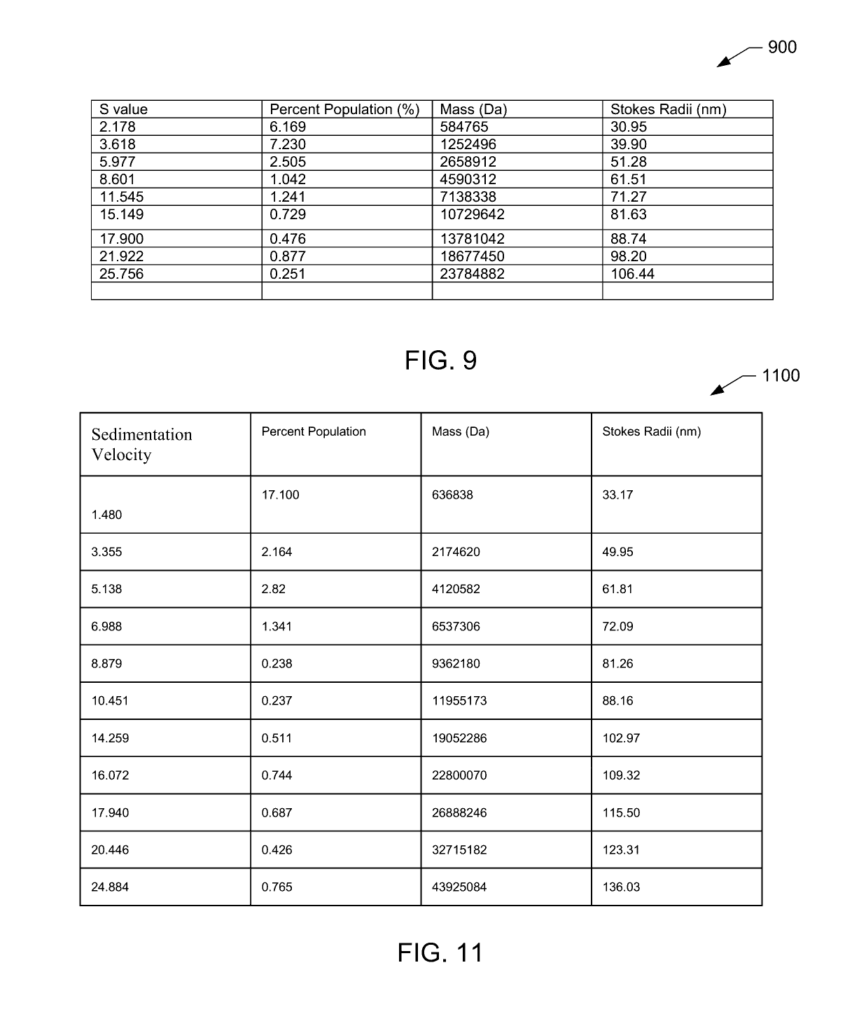

FIG. 9 shows a diagram of a table 900 that includes particle or fraction characterization data determined from the graphs 802 to 806, according to an example embodiment of the present disclosure. The column labeled ‘S value’ corresponds to the sedimentation coefficient determined from the curve peaks in graph 806. The column labeled ‘percent population (%)’ corresponds to a concentration of the particles that have the specified sedimentation coefficient. The ‘percent population (%)’ value corresponds to the area under the curve for each sedimentation coefficient. The column labeled ‘Mass’ corresponds to a calculated mass (in Da) of the particles for each sedimentation coefficient. The column labeled ‘Stokes Radii’ corresponds to a size (in nm) of the particles for each sedimentation coefficient. Together, the data in graph 900 provides a mass distribution for the measured particles, which at 260 nm, indicates the mass distribution of nucleic acid. It should be appreciated from the data in FIGS. 8 and 9 that the laboratory instrument 102 enables the particles to be distinguished having a size as low as 7 nm.

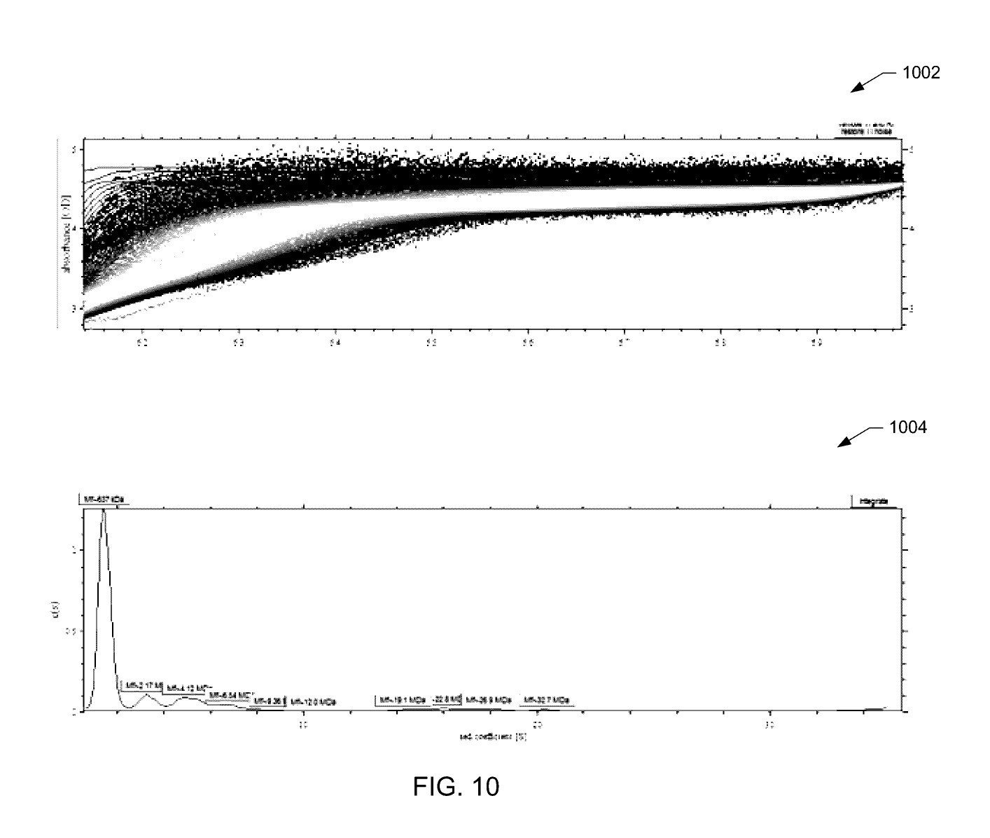

FIGS. 10 and 11 show a characterization of human serum H196 performed by the laboratory instrument 102, according to an example embodiment of the present disclosure. FIG. 10 shows a diagram of graph 1002 of the absorbance of the human serum at 210 nm. Graph 1004 shows the sedimentation coefficient and mass distribution of human serum. In this embodiment, no absorbance was determined at 260 nm or 280 nm, thereby indicating the human serum includes only cholesterol rafts, not exosomes. Table 1100 of FIG. 11 includes particle or fraction characterization data of human serum determined from the graphs 1002 and 1004.

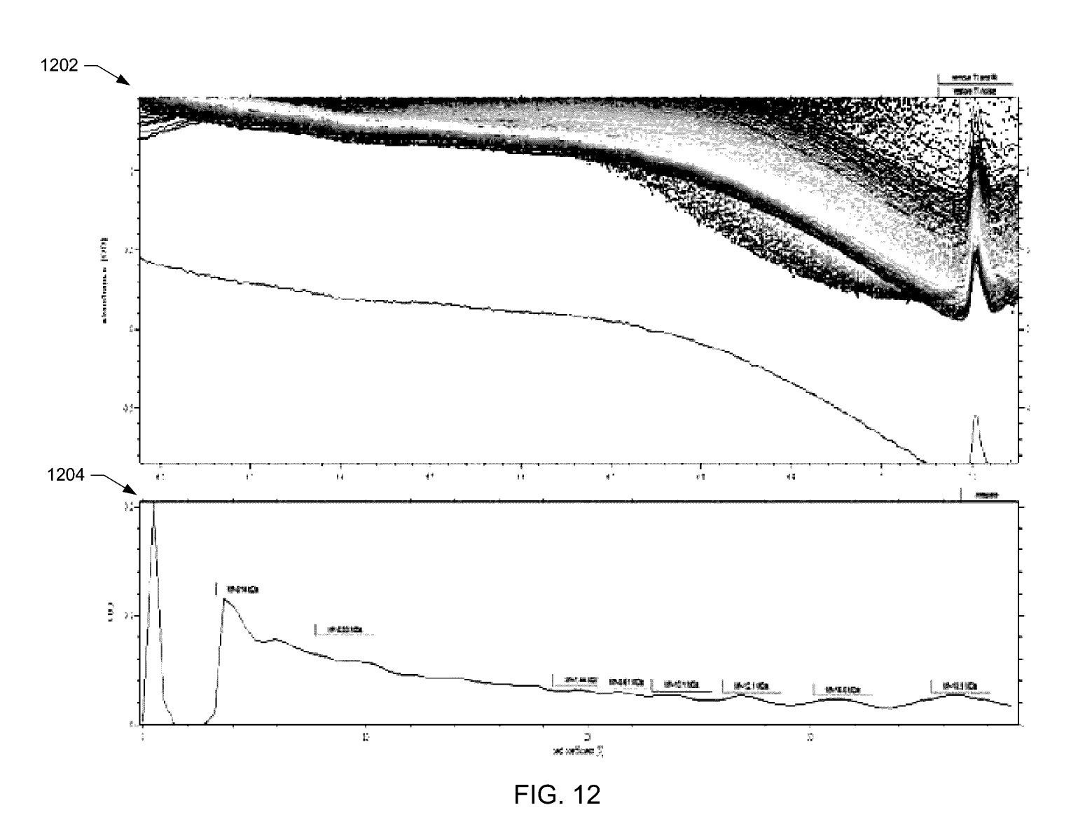

FIG. 12 shows a characterization of F50105 and F50102 liposomes performed by the laboratory instrument 102, according to an example embodiment of the present disclosure. Graph 1202 shows an absorbance measurement at 210 nm. Graph 1204 shows a curve for the sedimentation coefficients of the liposomes. As illustrated, the example laboratory instrument 102 is capable of determining the presence of liposomes and accordingly characterizing the liposomes based on mass distribution, percent population, and Stokes radii.

### Fluorescence Measurement Embodiments

As mentioned above, the example laboratory instrument 102 may also be configured to perform one or more absorbance measurements using fluorescence markers added to a sample solution. To perform an absorbance measurement using fluorescence markers, the laboratory instrument 102 uses the same (or similar) equipment used to perform the absorbance measurement described in conjunction with FIG. 5. The fluorescent markers may include, but are not limited to, pkh26 and/or pkh67. Pkh26 is configured to absorb light at 550 nm and pkh67 is configured to absorb light at 490 nm.

The use of fluorescent markers may enhance the detection of lipids, proteins, and/or nucleic acids. For example, some species of lipids, proteins, and/or nucleic acids may have a relatively low absorbance for any wavelength. However, these species may be bonded with a fluorescent marker, which may absorb relatively more light, albeit at a different wavelength. For instance, the detection of fluorescence absorption at either 490 nm or 550 nm indicates the presence of exosomes. Similar to non-fluorescence absorption, the level of absorption and/or sedimentation velocity is used to determine mass distribution, concentration, and Stokes radii of stained exosomes.

In some instances, the fluorescence-based absorbance measurements may be performed in conjunction with non-fluorescence absorbance measurements. For example, a sample solution may include different species or types of exosomes. Fluorescent markers may be added that bind to only one type of exosome. Absorbance measurements may be run to determine exosome purity of the non-marked exosome based on results from 210 nm, 260 nm, and 280 nm measurements. Fluorescence-based absorbance measurements may then be run to determine exosome purity of marked exosomes based on results from the 490 nm and 550 nm measurements.

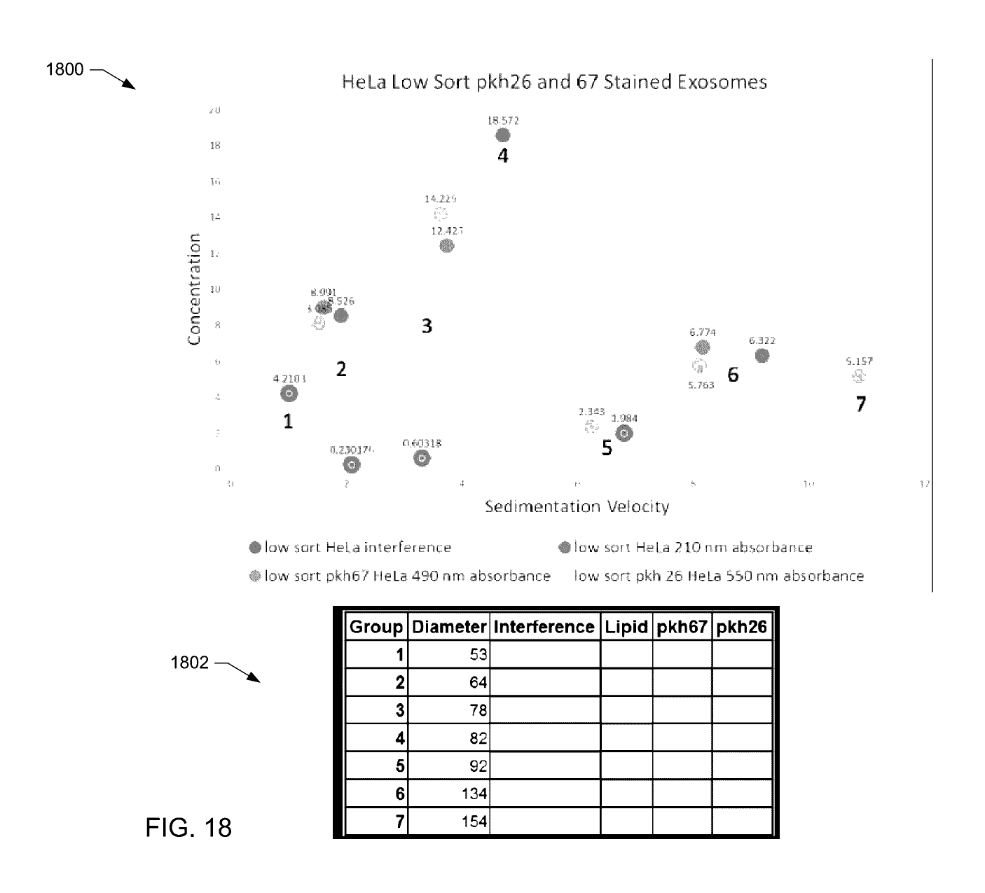

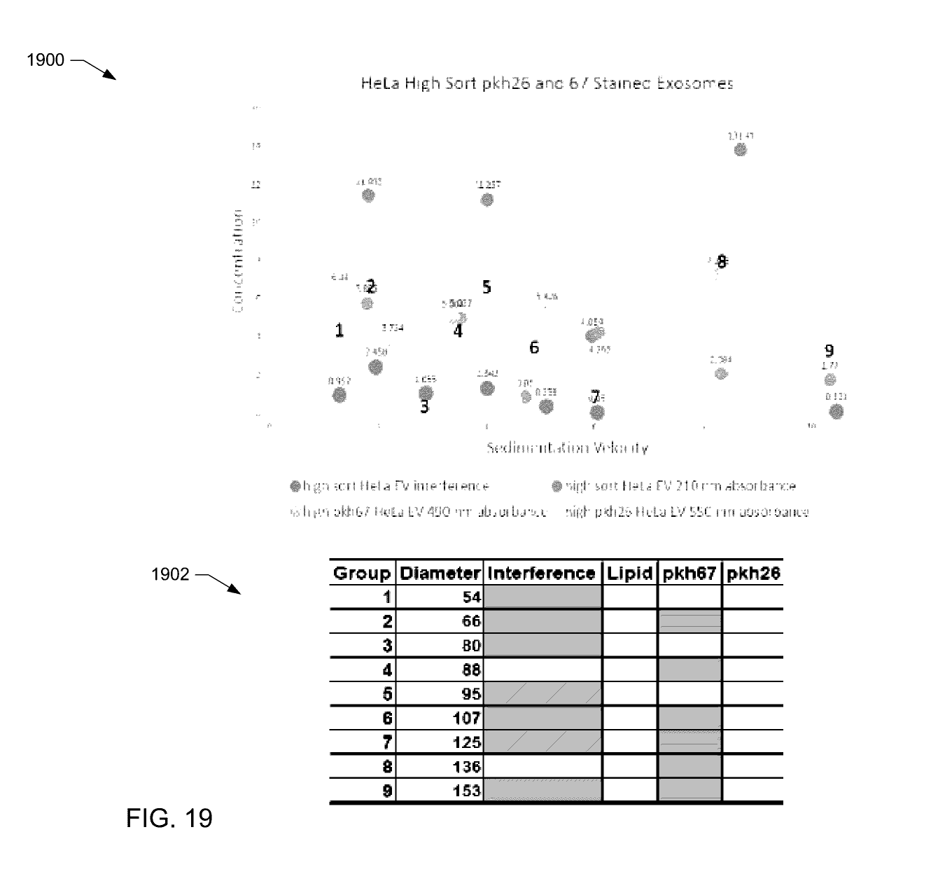

FIGS. 18 and 19 show diagrams of a characterization of HeLa exosomes isolated in a solution using flow-cytometry sorting, according to an example embodiment of the present disclosure. In this embodiment, the mass of the exosomes were determined (by determining sedimentation velocity and concentration) using an interference measurement, an absorbance measurement at 210 nm, and absorbance measurements using fluorescence markers at 490 nm and 550 nm. The data from the absorbance measurements was also used to characterize at least some of the measured particles as HeLa exosomes based on positive absorbance measurements at 210 nm (indicating the presence of lipids) and positive absorbance measurements at 490 nm and 550 nm using fluorescence markers (indicating the presence of pkh26 and pkh67 stains).

Graphs 1800 and 1900 show distributions of particle size in relation to concentration and sedimentation velocity, as determined by each of the different measurements. Tables 1802 and 1902 summarize positive identifications of species type for the different groups of particles. The characterization shown in FIG. 18 indicates the HeLa exosomes are present in groups 2 and 6, which correspond to particles having a diameter of 64 nm and 134 nm. Additionally, the characterization shown in FIG. 19 indicates the HeLa exosomes are present in groups 2 and 7 and 8, which correspond to particles having a diameter of 66 nm, 125 nm, and 136 nm. The other groups may include particles classified as liposomes, DNA, RNA, etc.

It should be appreciated that in some examples, measurements may be performed using the laboratory instrument 102 discussed above in conjunction with FIG. 5 to detect fluorescence emission. For example, a sample may include a target-specific fluorescence dye configured to couple to an antibody. Excitation light may be provided by the laboratory instrument 102, causing bonded fluorescence markers to emit light at one or more wavelengths. The emission data may be used to determine, for example, a sedimentation velocity, concentration, mass, mass distribution, and radius (e.g., Stokes Radius) for a plurality of particles or fractions within the marked solution.

### Interference Measurement Embodiments

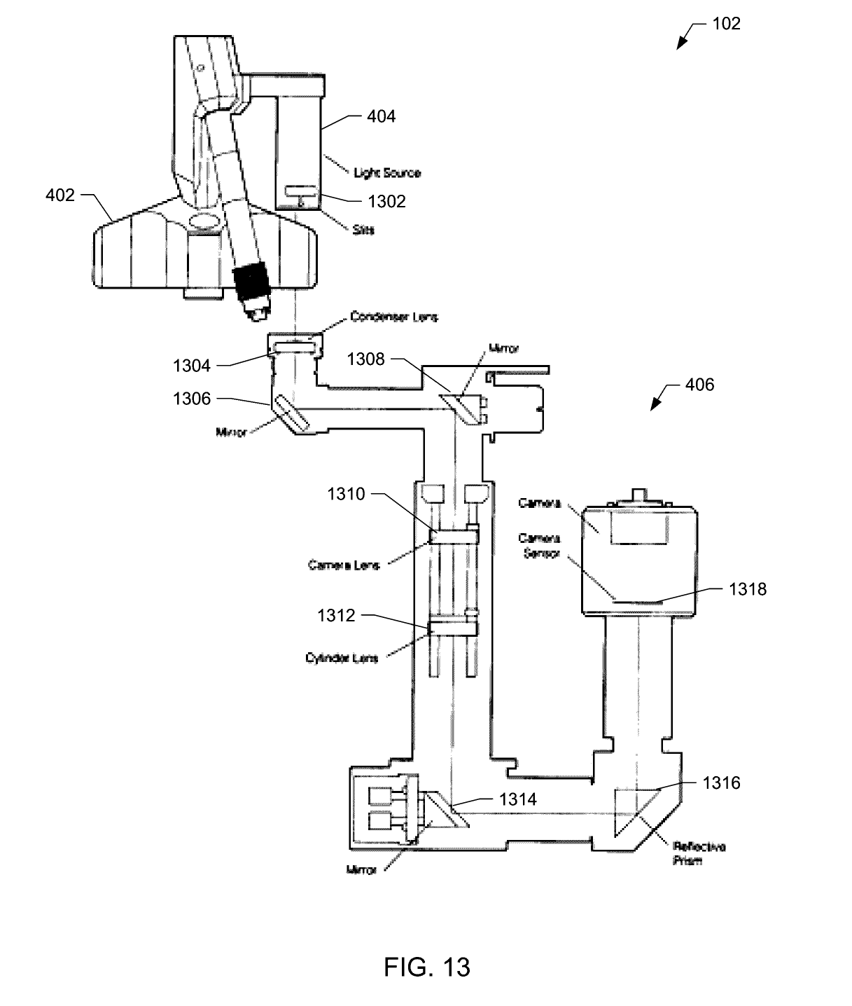

FIG. 13 shows a diagram of the laboratory instrument 102 configured to perform an interference measurement, according to an example embodiment of the present disclosure. In an interference measurement, parallel light waves are passed through the reference side 516 and the sample side 518 of the analytical cell 302 and reflected to cross, thereby producing an interference pattern. Positions of the resulting fringe pattern shift vertically in proportion to a concentration difference between the sample and reference sides of the cell 302. Shifts in the fringe pattern indicate a location of the sedimentation boundary 303.

In the example of FIG. 13, the light source 404 includes a diode laser configured to produce laser light. A collimating lens partitions the laser light into two parallel beams, which are passed through respective slits 1302 to the analytical cell 302. One or more lenses are configured to cross the light from the analytical cell and focus the light for the camera 406. In particular, a condensing lens 1304 causes the parallel light from the reference side 516 and the sample side 518 to cross. Mirrors 1306 and 1308 reflect the light to a camera lens 1310, which causes the interference pattern from the crossed light to be projected as parallel light waves. A cylinder lens 1312, mirror 1314, and reflective prism 1316 are configured to focus the interference or fringe pattern onto a camera sensor 1318 of the camera 406. Since the laser light is coherent, the resulting fringe pattern across the length of the analytical cell 302 may be recorded in a single image, thereby eliminating the need for moving parts.

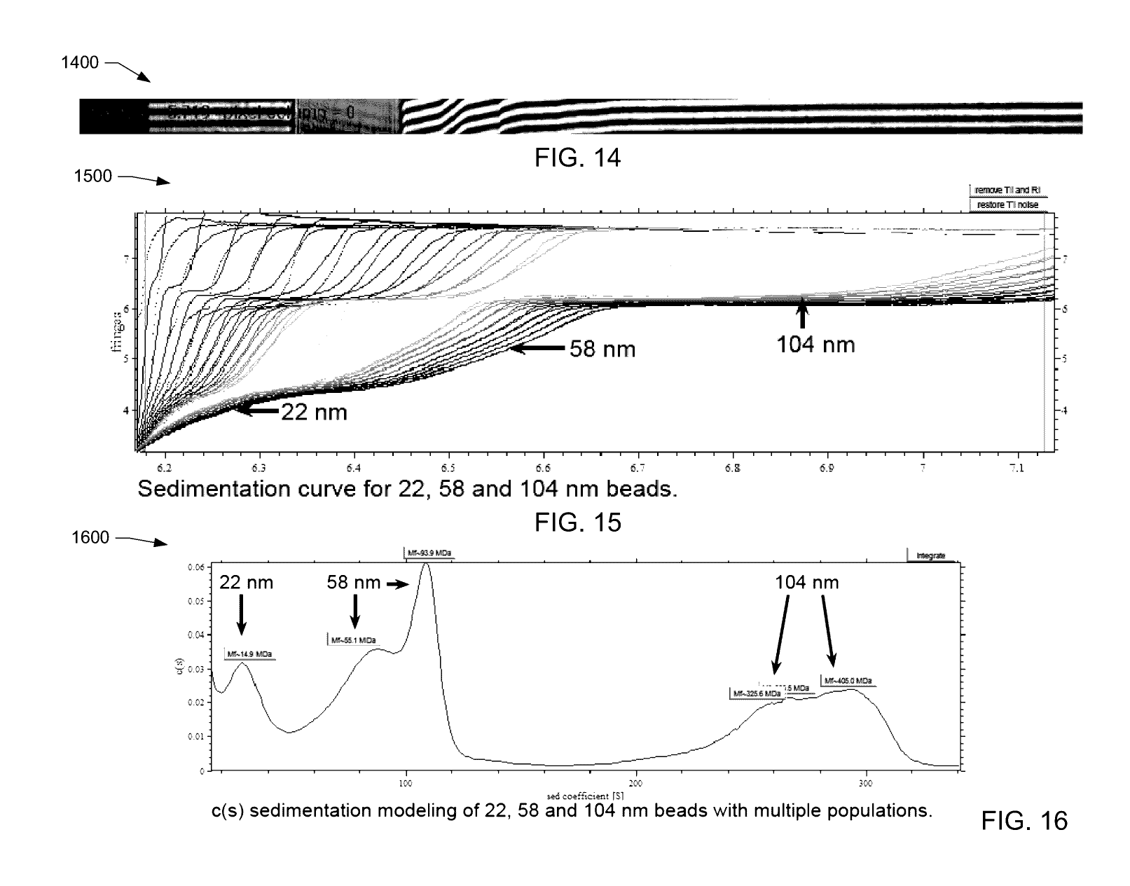

FIG. 14 shows a diagram of an example fringe pattern 1400, according to an example embodiment of the present disclosure. The vertical changes to the fringe pattern 1400 are indicative of the sedimentation boundary 303, which may be used to determine a sedimentation velocity. The fringe pattern 1400 was measured for latex polystyrene beads of various sizes (e.g., 22 nm, 58 nm, 104 nm). FIG. 15 shows a graph 1500 including a sedimentation curve for fringe patterns recorded over a time period from the same sample solution. The graph 1500 illustrates how the sedimentation boundary changes over time. FIG. 16 shows a graph 1600 of sedimentation coefficients determined from the sedimentation curve of graph 1500. The graph 1600 illustrates a mass distribution of the different sized beads. The concentration and Stokes radii may also be determined from the graph 1600.

### Exosome Characterization Embodiment

In some embodiments, a combination of the different measurements may be performed to obtain a highly accurate characterization of particles or fractions within a solution. Interference is typically more accurate (especially for solutions that sediment quickly) and faster at determining a mass distribution of particles since only a single scan has to be performed. Additionally, interference typically has a lower solution concentration requirement, for example, of about 5 ug/ml compared to 100 ug/ml for absorption (for bovine serum albumin (“BSA”). However, interference measurements cannot determine species or components of particles or fractions because interference cannot identify which particles absorb a specific wavelength of light. Accordingly, absorbance measurements may be performed to determine exosome purity based on contents of lipids, nucleic acid, and proteins.

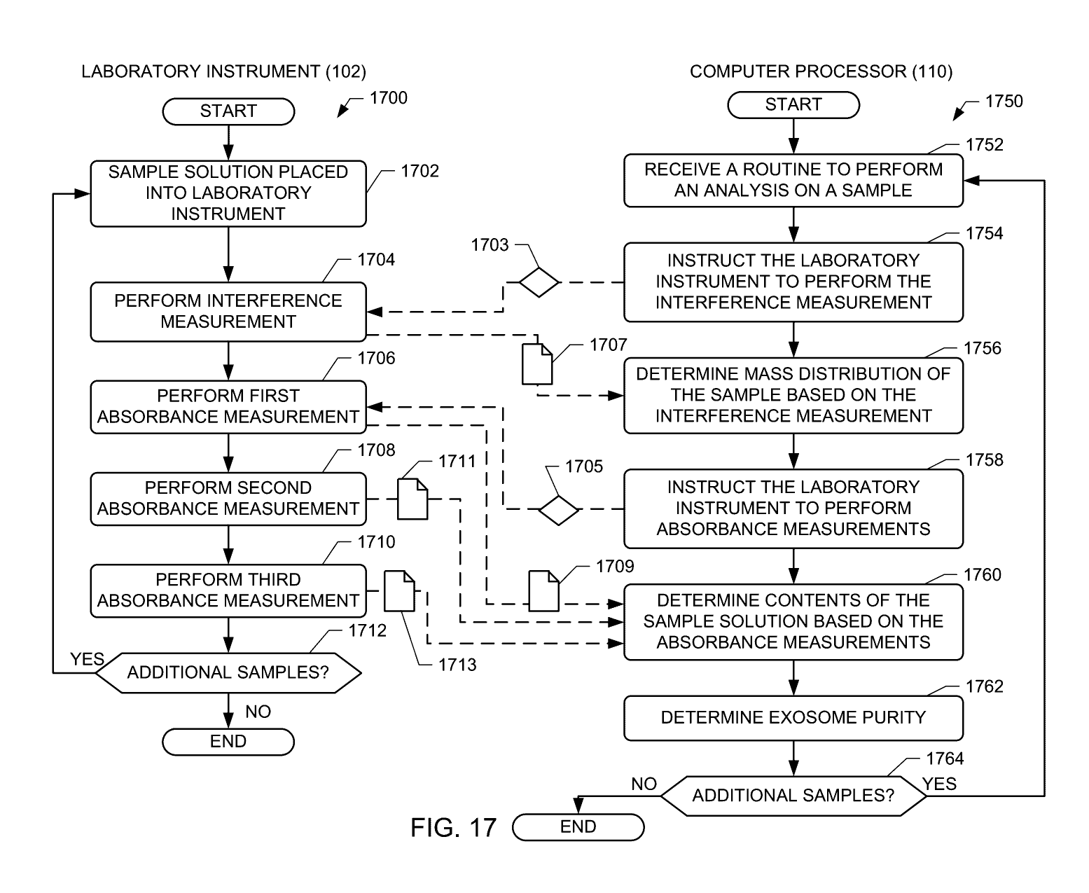

FIG. 17 illustrates a flow diagram showing example procedures 1700 and 1750 to determine exosome purity, according to an example embodiment of the present disclosure. Although the procedures 1700 and 1750 are described with reference to the flow diagrams illustrated in FIG. 17, it should be appreciated that many other methods of performing the steps associated with the procedures 1700 and 1750 may be used. For example, the order of many of the blocks may be changed, certain blocks may be combined with other blocks, and many of the blocks described are optional. For example, only some of the referenced interference and/or absorbance measurements may be needed to be performed to determine exosome purity. Further, the actions described in procedures 1700 and 1750 may be performed among multiple devices.

The example procedure 1700 of FIG. 17 operates on, for example, the laboratory instrument 102 of FIGS. 1, 4, 5, and 13. The procedure 1700 begins when a sample solution is placed into one or more analytical cells (block 1702). In some instances, the sample solution may include particles and/or fractions that have already been isolated and/or sorted. For example, the Beckman Coulter® MoFlo® Astrios™ EQ sorter may sort fractions prior to the fractions being added to the analytical cell for analysis. Additionally, in some instances, the routine and/or specification may instead be entered via a user interface at the laboratory instrument 102.

After receiving a routine or specification to perform measurements on the analytical cells, the laboratory instrument 102 is configured to perform the appropriate measurements based on the description provided above in conjunction with FIGS. 1 to 7 and 13 to 15. In some instances, the laboratory instrument 102 may receive a first routine 1703 to perform the interference measurement and a second routine 1705 to perform the absorbance measurement(s). In other instances, a single routine is received prior to beginning the first measurement. The laboratory instrument 102 then preforms the measurements including, for example, an interference measurement (block 1704), a first absorbance measurement at 210 nm (block 1706), a second absorbance measurement at 260 nm (block 1708), and a third absorbance measurement at 280 nm (block 1710). The laboratory instrument is configured to transmit results 1707, 1709, 1711, and 1713 after (or during) the respective measurements. In some instances, the laboratory instrument 102 may need to be reconfigured to transition from performing the interference measurement to the absorbance measurements. It should be appreciated that one or more fluorescence measurements may be performed in addition to (or alternatively to) the absorbance measurements.

The laboratory instrument 102 may then determine whether additional samples are to be measured (block 1712). Conditioned upon determining at least one additional sample is to be measured, the laboratory instrument 102 receives another sample and performs the appropriate measurements (blocks 1702 to 1710). However, conditioned upon determining there are no additional samples to measure, the example procedure 1700 ends.

The example procedure 1750 of FIG. 17 operates on, for example, the computer processor 110 of FIGS. 1 and 4. The procedure 1750 begins when the computer processor 110 receives from a user a routine, instructions, or an algorithm to perform an analysis on a sample (block 1752). The computer processor 110 then transmits one or more messages including the routine 1703 to perform an interference measurement (block 1754). Alternatively, the routine or instructions may instead be entered directly at the laboratory instrument 102. The computer processor 110 next receives interference measurement data 1707 from the laboratory instrument 102 after the laboratory instrument has been configured for and performs the interference measurement. The computer processor 110 uses the interference measurement data 1707 to determine, for example, a mass distribution, concentration, and/or Stokes radii for particles and/or fractions with the sample (block 1756).

The computer processor 110 then transmits one or more messages including the routine 1705 to perform one or more absorbance (and/or fluorescence) measurements (block 1758). The computer processor 110 receives absorbance measurement data 1709, 1711, and 1713 from the laboratory instrument 102 after the laboratory instrument has been configured for and performs the absorbance measurement(s). The computer processor 110 uses the absorbance measurement data 1709, 1711, and 1713 to determine, for example, lipid content, protein content, and nucleic acid (block 1760). The example computer processor 110 may then determine exosome purity based on the interference and absorbance-based characterization of the particles and/or fractions (block 1762).

The computer processor 110 may then determine whether additional samples are to be measured (block 1764). Conditioned upon determining at least one additional sample is to be measured, the computer processor 110 receives another routine (or uses the same routine) and performs the appropriate analysis (blocks 1752 to 1762). However, conditioned upon determining there are no additional samples to measure, the example procedure 1750 ends.

### CONCLUSION

It will be appreciated that all of the disclosed methods and procedures described herein can be implemented using one or more computer programs or components. These components may be provided as a series of computer instructions on any computer-readable medium, including RAM, ROM, flash memory, magnetic or optical disks, optical memory, or other storage media. The instructions may be configured to be executed by a processor, which when executing the series of computer instructions performs or facilitates the performance of all or part of the disclosed methods and procedures.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the example disclosed methods and apparatus (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the example disclosed methods and apparatus and does not pose a limitation on the scope of the example methods and apparatus otherwise claimed. No language in the specification should be construed as indicating any non-claimed element essential to the practice of the example disclosed methods and apparatus.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments of the example methods and apparatus disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of example methods and apparatus are described herein, including the best mode known to the inventors for carrying out the example methods and apparatus. Of course, variations on those embodiments will become apparent to those of ordinary skill in the art upon reading the foregoing description. The applicant expects those of ordinary skill in the art to employ such variations as appropriate, and the applicant intends for the example methods and apparatus to be practiced otherwise than specifically described herein. Accordingly, the example methods and apparatus disclosed herein include all modifications and equivalents of the subject matter recited in the claims appended hereto as permitted by applicable law. Moreover, any combination of the above-described elements in all possible variations thereof is encompassed by the example disclosed methods and apparatus unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the example disclosed methods and apparatus so claimed are inherently or expressly described and enabled herein.

Further, it is to be understood that the embodiments of the example methods and apparatus disclosed herein are illustrative of the principles of the present methods and apparatus. Other modifications that may be employed are within the scope of the example methods and apparatus. Thus, by way of example, but not of limitation, alternative configurations of the present methods and apparatus may be utilized in accordance with the teachings herein. Accordingly, the present example disclosed methods and apparatus are not limited to that precisely as shown and described.

The invention is claimed as follows:

1. A laboratory instrument apparatus comprising:
an analytical centrifuge configured to:
rotate a solution causing a plurality of particles to separate, and
perform at least one of:
i) an interference measurement on the solution, and
ii) three absorbance measurements on the solution; and
a computer processor communicatively coupled to the analytical centrifuge configured to determine an exosome purity of the solution based on the at least one of the three absorbance measurements and the interference measurement.

2. The apparatus of claim 1, wherein the computer processor is configured to use the interference measurement to determine a mass distribution of the plurality of particles.

3. The apparatus of claim 1, wherein the computer processor is configured to use the interference measurement to determine at least one isolated species within the plurality of particles.

4. The apparatus of claim 1, wherein the computer processor is configured to use the interference measurement to determine that at least some of the plurality of particles include exosomes.

5. The apparatus of claim 1, wherein the analytical centrifuge is configured to:
perform a first absorbance measurement on the solution using light with a wavelength between 180 nm and 230 to determine lipid content;
perform a second absorbance measurement on the solution using light with a wavelength between 230 nm and 270 nm to determine nucleic acid content; and
perform a third absorbance measurement on the solution using light with a wavelength between 270 nm and 300 nm to determine protein content.

6. The apparatus of claim 5, wherein the computer processor is configured to determine at least some of the plurality of particles are exosomes conditioned on detecting lipid content and at least one of nucleic acid content and protein content.

7. The apparatus of claim 5, wherein the computer processor is configured to determine the exosome purity based on a ratio of the lipid content, nucleic acid content, and protein content.

8. The apparatus of claim 5, wherein the computer processor is configured to determine at least some of the plurality of particles are lipid rafts conditioned on detecting only lipid content.

9. The apparatus of claim 1, wherein the computer processor is configured to characterize at least some of the particles as at least one of extracellular vesicles and microvesicles based on the three absorbance measurements or the interference measurement.

10. A method of characterizing extracellular vesicles in a sample solution, the method comprising:
rotating the sample solution using an analytical centrifuge to separate the sample solution into a plurality of fractions;
measuring with the analytical centrifuge a first interaction of the plurality of fractions with a first energy beam;
measuring with the analytical centrifuge a second interaction of the plurality of fractions with a second energy beam; and
characterizing, via a computer processor, the extracellular vesicles along the plurality of fractions based on the first interaction and the second interaction.

11. The method of claim 10, further comprising determining, via the computer processor, a size distribution of the extracellular vesicles based on at least one of the first interaction and the second interaction.

12. The method of claim 10, further comprising:
determining at least one sedimentation velocity for the plurality of fractions; and
determining, via the computer processor, a size distribution of the extracellular vesicles based on at least one of the first interaction and the second interaction in conjunction with the at least one sedimentation velocity.

13. The method of claim 10, wherein at least one of the first interaction and the second interaction includes an absorbance measurement.

14. The method of claim 10, wherein at least one of the first energy beam and the second energy beam has a wavelength between 150 nm and 400 nm.

15. The method of claim 10, wherein at least one of the first interaction and the second interaction include a fluorescence measurement.

16. The method of claim 15, further comprising adding a nucleic-acid-binding dye to the sample solution, wherein the fluorescence measurement is made from a labeled nucleic-acid probe.

17. The method of claim 15, wherein the fluorescence measurement is made from a dye-labeled antibody.

18. A machine-accessible device having instructions stored thereon that are configured when executed to cause a machine to at least:
instruct an analytical centrifuge to rotate a solution to cause a plurality of particles to separate;
instruct the analytical centrifuge to perform a first measurement on the solution using a first energy beam;
instruct the analytical centrifuge to perform a second measurement on the solution using a second energy beam;
determine a mass distribution of the plurality of particles based on at least one of the first measurement and the second measurement;
determine an exosome purity of the plurality of particles based on at least one of the first measurement and the second measurement; and
create for display a graphical representation of at least one of the mass distribution and the exosome purity.

19. The machine-accessible device of claim 18, further comprising instructions stored thereon that are configured when executed to cause a machine to determine the exosome purity by identifying, within at least some of the plurality of particles, lipids and at least one of nucleic acids and proteins.

20. The machine-accessible device of claim 18, further comprising instructions stored thereon that are configured when executed to cause a machine to determine the exosome purity by determining a ratio of lipid content, nucleic acid content, and protein content.
